SEQUENCE LISTING (30 sequences)

SEQ ID NO: 1 - DNA, 19 bp, Artificial Sequence
  Primer
tcacttgcttccgttgagg

SEQ ID NO: 2 - DNA, 20 bp, Artificial Sequence
  Primer
ggtttcggatgttacagcgt

SEQ ID NO: 3 - DNA, 47 bp, Artificial Sequence
  Primer
cacagctttcacttgcttccgttgagggttcaagcctccaagctgtg

SEQ ID NO: 4 - DNA, 48 bp, Artificial Sequence
  Primer
agaactccggtttcggatgttacagcgtctgcgaggcgagggagttct

SEQ ID NO: 5 - DNA, 50 bp, Artificial Sequence
  Primer
catagctcacttgcttccgttgaggttttgctcctctttaccatgctatg

SEQ ID NO: 6 - DNA, 51 bp, Artificial Sequence
  Primer
caaagaggtttcggatgttacagcgtggaaatgtctcaggtactttctttg

SEQ ID NO: 7 - DNA, 45 bp, Artificial Sequence
  Primer
acggtctcacttgcttccgttgaggaacattccgaaggggaccgt

SEQ ID NO: 8 - DNA, 44 bp, Artificial Sequence
  Primer
cgtcctggtttcggatgttacagcgtggcatccgaaggaggacg

SEQ ID NO: 9 - DNA, 43 bp, Artificial Sequence
  Primer
cattatgtcacttgcttccgttgaggcccacgcgcgcataatg

SEQ ID NO: 10 - DNA, 44 bp, Artificial Sequence
  Primer
ctagggtttcggatgttacagcgtttcacttcggtctcccctag

SEQ ID NO: 11 - DNA, 19 bp, Artificial Sequence
  Primer
tcacttgcttccgttgagg

SEQ ID NO: 12 - DNA, 20 bp, Artificial Sequence
  Primer
ggtttcggatgttacagcgt

SEQ ID NO: 13 - DNA, 54 bp, Artificial Sequence
  Primer
tcacttgcttccgttgagggaagatctagacagtggatacataacaaatgcatg

SEQ ID NO: 14 - DNA, 55 bp, Artificial Sequence
  Primer
ggtttcggatgttacagcgtttctccgaaggtaattgcctcccagatctgagtcc

SEQ ID NO: 15 - DNA, 44 bp, Artificial Sequence
  Primer
tcacttgcttccgttgaggtcatccatcatcttcggcagattaa

SEQ ID NO: 16 - DNA, 50 bp, Artificial Sequence
  Primer
ggtttcggatgttacagcgtcaggcggtagagtatgccaaatgaaaatca

SEQ ID NO: 17 - DNA, 48 bp, Artificial Sequence
  Primer
tcacttgcttccgttgagggaacatgtcaaagtcactggacttcatgg

SEQ ID NO: 18 - DNA, 49 bp, Artificial Sequence
  Primer
ggtttcggatgttacagcgtatatatgtgttacctacccttgtcggtcc

SEQ ID NO: 19 - DNA, 47 bp, Artificial Sequence
  Primer
tcacttgcttccgttgaggcaccaaatggattaagatgttcatgaat

SEQ ID NO: 20 - DNA, 48 bp, Artificial Sequence
  Primer
ggtttcggatgttacagcgttctctctcacccagtcatcacttcatag

SEQ ID NO: 21 - DNA, 48 bp, Artificial Sequence
  Primer
tcacttgcttccgttgaggaataggagtacctgagatgtagcagaaat

SEQ ID NO: 22 - DNA, 48 bp, Artificial Sequence
  Primer
ggtttcggatgttacagcgtctgaccttaagttgttcttccaaagcag

SEQ ID NO: 23 - DNA, 46 bp, Artificial Sequence
  Primer
tcacttgcttccgttgaggccacatgtaggtcaaaaatgtaatgaa

SEQ ID NO: 24 - DNA, 51 bp, Artificial Sequence
  Primer
ggtttcggatgttacagcgtgtctcagtaatcttcttacctatgactatgg

SEQ ID NO: 25 - DNA, 47 bp, Artificial Sequence
  Primer
tcacttgcttccgttgaggcgttgttgcatttgtctgtttcagttac

SEQ ID NO: 26 - DNA, 47 bp, Artificial Sequence
  Primer
ggtttcggatgttacagcgtgtctaacctttatccactggagatttg

SEQ ID NO: 27 - DNA, 48 bp, Artificial Sequence
  Primer
tcacttgcttccgttgaggaggagaaattgcgcctctgaaagagaacg

SEQ ID NO: 28 - DNA, 48 bp, Artificial Sequence
  Primer
ggtttcggatgttacagcgtctgcagaagcttccatctggtgttcagg

SEQ ID NO: 29 - DNA, 46 bp, Artificial Sequence
  Primer
tcacttgcttccgttgaggaatgcaggatttggaacagaggcgtcc

SEQ ID NO: 30 - DNA, 48 bp, Artificial Sequence
  Primer
ggtttcggatgttacagcgtttcgatccgtaatgattgttctagcctc

(19) United States
(12) Patent Application Publication
(10) Pub. No.: US 2007/0059700 A1
(43) Pub. Date: Mar. 15, 2007

(54) Methods and compositions for optimizing multiplex pcr primers

(76) Inventors: Shengce Tao, Beijing (CN); Jing Cheng, Beijing (CN)

Correspondence Address: MORRISON & FOERSTER LLP, 12531 HIGH BLUFF DRIVE, SUITE 100, SAN DIEGO, CA, 92130-2040, US

(21) Appl. No.: 10/559,951

(22) PCT Filed: May 9, 2003
(86) PCT No.: PCT/CN03/00335

§ 371 Date: Jun. 27, 2006

(51) Int. Cl. C12Q 1/68

(52) U.S. Cl. 435/6

(57) ABSTRACT

This invention relates generally to the field of PCR. In particular, the invention provides methods, compositions and kits for optimizing multiplex PCR primers using, inter alia, a plurality of 5′ and 3′ specific primers and a 5′ and a 3′ universal primer at various ratios.

### BACKGROUND OF THE INVENTION

Multiplex PCR is a way of PCR amplification which uses multiple pairs of primers to amplify multiple target seqence simultaneously in a single reaction tube. Use of multiplex PCR can significantly simplify experimental procedures in nucleic acid analysis and detection and shorten the time used. In addition, multiplex PCR requires no additional procedures and equipment. After first reported in 1988 (Chamberlain, J. S., et al., 1988. Nucleic Acids Res., 16, 11141-11156), multiplex PCR becomes a fast and simple screening method for clinical and research laboratories. Multiplex PCR has been successfully used in areas including gene deletion analysis (Sieber, O. M., et al., 2002. Proc. Natl. Acad. Sci. U.S.A. 99, 2954-2958), gene mutation and gene polymorphism analysis (Moutou, C., et al., 2002. Eur. J. Hum. Genet. 10, 231-238), quantitative analysis of mRNA (Zimmermann, K., et al., 1996. Biotechniques 21, 480-484), RNA detection (Jin, L., et al., 1996. Mol. Cell Probes, 10, 191-200; Zou, S., et al., 1998. J. Clin. Microbiol., 36, 1544-1548), and gene sequence analysis (Tettelin, H., et al., 1999. Genomics, 62, 500-507). In the area of diagnosis of infectious diseases, multiplex PCR already plays a significant role in identification and analytical research of virus (Druce, J., et al., 2002. J. Clin. Microbiol., 40, 1728-1732; Robert, P. Y., et al., 2002. J. Med. Virol., 66, 506-511), bacteria (Osek, J., 2002. Lett. Appl. Microbiol., 34, 304-310; Sloan, L. M., et al., 2002. J. Clin. Microbiol., 40, 96-100), parasite (Harris, E., et al., 1998. J. Clin. Microbiol., 36, 1989-1995), and drug tolerance of bacteria.

Very detailed primer design and multi-screening processes are usually required to establish an effective multiplex PCR program. Problems often encountered in multiplex PCR are the imbalance of different target fragments amplified (some of the target fragments may not effectively amplified at all) and relative low reproducibility. The following factors should be considered for a successful multiplex PCR program: the balance between concentrations of primers used, PCR buffer, Magnesium, and dNTP, temperature of each step in PCR cycle, and the amount of template DNA and Taq DNA polymerase used. The optimization of denaturing temperature in PCR and buffer system, the ratio of Magnesium and dNTP concentration, and concentration of primers are necessary for specificity of multiplex PCR. See Markoulatos, P., et al., 2002. J. Clin. Lab Anal., 16, 47-51; Elnifro, E. M., et al., 2000. Clin. Microbiol. Rev., 13, 559-570; and Henegariu, O., et al., 1997. Biotechniques, 23, 504-511. After studies of factors involved in multiplex PCR, a detailed optimizing program can be developed which requires multiple steps of optimization.

A common phenomenon in multiplex PCR is amplification of one or some of sequences resulting that the ratio of PCR products is different or even significantly different from the ratio of starting template. This is caused by the limitation of polymerase and dNTP in the PCR system. The primers in the PCR system compete for the limited polymerase and dNTP and the amplification efficiencies of these primers are different. Selection of primers, not the template, is important for the imbalance of multiplex PCR products (Suzuki, M. T. and Giovannoni, S. J. 1996. Appl. Environ. Microbiol., 62, 625-630). Thus, determination of the final concentration of each primer becomes a key factor for a multiplex PCR system. To avoid cross hybridization between primers and nonspecific template, each primer should be carefully designed and analyzed. For an ideal multiplex PCR, all primers should have the same amplification efficiency. The same efficiency of different primers can be established by designing similar Tm for each primer (e.g., length of primer between 18 to 28 nucleotides, and GC content between 45 to 60%, and no homology between primers and no self homology). Optimizing concentration of each primer in multiplex PCR can be achieved by experiment.

### BRIEF SUMMARY OF THE INVENTION

In one aspect, the present invention is directed to a method for optimizing multiplex PCR primers, which method comprises: a) providing a plurality of 5′ and 3′ specific primers, each of said specific primers comprising a specific sequence complementary to its target sequence to be amplified and a common sequence; b) providing a 5′ and a 3′ universal primer, said 5′ universal primer being complementary to said common sequence of said 5′ specific primers and said 3′ universal primer being complementary to said common sequence of said 3′ specific primers; c) conducting a plurality of multiplex PCRs on a plurality of target sequences in the presence of said plurality of 5′ and 3′ specific primers and said 5′ and 3′ universal primers, wherein in each of said PCRs, the concentration of said 5′ and 3′ universal primers equals to or is higher than the concentration of said 5′ and 3′ specific primers, respectively, and the concentrations of said 5′ and 3′ specific primers in different PCRs are different, respectively; and d) assessing PCR products of said different PCRs and identifying a PCR wherein said target sequences are comparably amplified to identify optimized multiplex PCR primers for amplifying said target sequences in multiplex PCRs.

In another aspect, the present invention is directed to a composition for optimizing multiplex PCR primers, which composition comprises: a) a plurality of different concentrations of a plurality of 5′ and 3′ specific primers, each of said specific primers comprising a specific sequence complementary to its target sequence to be amplified and a common sequence; and b) a concentration of a 5′ and a 3′ universal primer, said 5′ universal primer being complementary to said common sequence of said 5′ specific primers and said 3′ universal primer being complementary to said common sequence of said 3′ specific primers, wherein the concentration of said 5′ and 3′ universal primer equals to or is higher than any of the concentrations of said 5′ and 3′ specific primers, respectively.

In still another aspect, the present invention is directed to a kit for optimizing multiplex PCR primers, which kit comprises: a) a plurality of 5′ and 3′ specific primers, each of said specific primers comprising a specific sequence complementary to its target sequence to be amplified and a common sequence; b) a 5′ and a 3′ universal primer, said 5′ universal primer being complementary to said common sequence of said 5′ specific primers and said 3′ universal primer being complementary to said common sequence of said 3′ specific primers; c) means for conducting a plurality of multiplex PCRs on a plurality of target sequences in the presence of said plurality of 5′ and 3′ specific primers and said 5′ and 3′ universal primers, wherein in each of said PCRS, the concentration of said 5′ and 3′ universal primers equals to or is higher than the concentration of said 5′ and 3′ specific primers, respectively, and the concentrations of said 5′ and 3′ specific primers in different PCRs are different, respectively; and d) means for assessing PCR products in said different PCRs and identifying a PCR wherein said target sequences are comparably amplified to identify optimized multiplex PCR primers for amplifying said target sequences in multiplex PCRS.

### BRIEF DESCRIPTION OF THE SEVERAL VIEWS OF THE DRAWING(S)

FIG. 1 illustrates an exemplary optimization procedure. 1 depicts an upstream universal primer; 2 depicts an upstream specific primer with a common sequence; 3 depicts a template; 4 depicts a downstream specific primer with a common sequence; and 5 depicts a downstream universal primer.

FIG. 2 illustrates another exemplary optimization procedure.

FIG. 3 illustrates amplification of HBV, HAV, HDV and EBV.

FIG. 4 illustrates amplification of DMD.

### DETAILED DESCRIPTION OF THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “polymerase chain reaction (PCR)” refers to a system for in vitro amplification of DNA. Two synthetic oligonucleotide primers, which are complementary to two regions of the target DNA (one for each strand) to be amplified, are added to the target DNA (that need not be pure), in the presence of excess deoxynucleotides and a heat-stable DNA polymerase, e.g., Taq DNA polymerase. In a series, e.g., 30, of temperature cycles, the target DNA is repeatedly denatured (e.g., around 90° C.), annealed to the primers (e.g., at 50-60° C.) and a daughter strand extended from the primers (e.g., 72° C.). As the daughter strands themselves act as templates for subsequent cycles, DNA fragments matching both primers are amplified exponentially, rather than linearly. The original DNA need thus be neither pure nor abundant, and the PCR reaction has accordingly become widely used not only in research, but in clinical diagnostics and forensic science.

As used herein, “nested PCR” refers to a PCR in which specificity is improved by using two sets of primers sequentially. An initial PCR is performed with the “outer” primer pairs, then a small aliquot is used as a template for a second round of PCR with the “inner” primer pair.

As used herein, “reverse transcription PCR or RT-PCR” refers to PCR in which the starting template is RNA, implying the need for an initial reverse transcriptase step to make a DNA template. Some thermostable polymerases have appreciable reverse transcriptase activity; however, it is more common to perform an explicit reverse transcription, inactivate the reverse transcriptase or purify the product, and proceed to a separate conventional PCR.

As used herein, “primer” refers to an oligonucleotide that hybridizes to a target sequence, typically to prime the nucleic acid in the amplification process.

As used herein, “the concentration of said 5′ and 3′ universal primers equals to or is higher than the concentration of said 5′ and 3′ specific primers, respectively” means that the concentration of the 5′ universal primer equals to or is higher than the concentration of the 5′ specific primers and the concentration of the 3′ universal primer equals to or is higher than the concentration of the 3′ specific primers.

As used herein, “the concentrations of said 5′ and 3′ specific primers in different PCRs are different, respectively” means that the concentrations of the 5′ specific primers in different PCRs are different from each other and the concentrations of the 3′ specific primers in different PCRs are different from each other.

As used herein, “said target sequences are comparably amplified” means that the degree of the amplification of the different target sequences in the multiplex PCR achieve the desired uniformity. Normally, the difference among the amplification level of different target sequences should be within about 50% from each other. Preferably, the difference among the amplification level of the different target sequences should be within about 40%, 30%, 20%, 10%, 5%, 4%, 3%, 2%, 1% from each other. More preferably, the different target sequences are amplified to the same degree.

As used herein, “hairpin structure” refers to a polynucleotide or nucleic acid that contains a double-stranded stem segment and a single-stranded loop segment wherein the two polynucleotide or nucleic acid strands that form the double-stranded stem segment is linked and separated by the single polynucleotide or nucleic acid strand that forms the loop segment. The “hairpin structure” can further comprise 3′ and/or 5′ single-stranded region(s) extending from the double-stranded stem segment.

As used herein, “nucleic acid (s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

As used herein, “complementary or matched” means that two nucleic acid sequences have at least 50% sequence identity. Preferably, the two nucleic acid sequences have at least 60%, 70%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

As used herein: “stringency of hybridization” in determining percentage mismatch is as follows:

1) high stringency: 0.1×SSPE (or 0.1×SSC), 0.1% SDS, 65° C.;

2) medium stringency: 0.2×SSPE (or 1.0×SSC), 0.1% SDS, 50° C. (also referred to as moderate stringency); and

3) low stringency: 1.0×SSPE (or 5.0×SSC), 0.1% SDS, 50° C.

It is understood that equivalent stringencies may be achieved using alternative buffers, salts and temperatures.

As used herein, “gene” refers to the unit of inheritance that occupies a specific locus on a chromosome, the existence of which can be confirmed by the occurrence of different allelic forms. Given the occurrence of split genes, gene also encompasses the set of DNA sequences (exons) that are required to produce a single polypeptide.

As used herein, “melting temperature” (“Tm”) refers to the midpoint of the temperature range over which nucleic acid duplex, i.e., DNA:DNA, DNA:RNA, RNA:RNA, PNA:DNA, LNA:RNA and LNA:DNA, etc., is denatured.

As used herein, “sample” refers to anything which may contain a target nucleic acid to be amplified by PCR, e.g., multiplex PCR. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood. Samples used for the present invention include such target-cell enriched cell preparation.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein, “assessing PCR products” refers to quantitative and/or qualitative determination of the PCR products, and also of obtaining an index, ratio, percentage, visual or other value indicative of the level of the PCR products. Assessment may be direct or indirect and the chemical species actually detected need not of course be the PCR products themselves but may, for example, be a derivative thereof, or some further substance.

### B. METHODS FOR OPTIMIZING MULTIPLEX PCR PRIMERS

In one aspect, the present invention is directed to a method for optimizing multiplex PCR primers, which method comprises: a) providing a plurality of 5′ and 3′ specific primers, each of said specific primers comprising a specific sequence complementary to its target sequence to be amplified and a common sequence; b) providing a 5′ and a 3′ universal primer, said 5′ universal primer being complementary to said common sequence of said 5′ specific primers and said 3′ universal primer being complementary to said common sequence of said 3′ specific primers; c) conducting a plurality of multiplex PCRs on a plurality of target sequences in the presence of said plurality of 5′ and 3′ specific primers and said 5′ and 3′ universal primers, wherein in each of said PCRS, the concentration of said 5′ and 3′ universal primers equals to or is higher than the concentration of said 5′ and 3′ specific primers, respectively, and the concentrations of said 5′ and 3′ specific primers in different PCRs are different, respectively; and d) assessing PCR products of said different PCRs and identifying a PCR wherein said target sequences are comparably amplified to identify optimized multiplex PCR primers for amplifying said target sequences in multiplex PCRs.

In a specific embodiment, at least one of the specific primers, or some or all of the specific primers can further comprise, at the 5′ end, a sequence complementary to a sequence at the 3′ end so that under suitable conditions, a hairpin structure is formed within the specific primer(s).

The 5′ and 3′ specific primers and the universal primers can be added together or separately in the PCR. For example, the 5′ and 3′ specific primers and the 5′ and a 3′ universal primers can be present in the multiplex PCRs simultaneously. In another example, the 5′ and 3′ universal primers are added into the multiplex PCRs after the amplification has been initiated with the 5′ and 3′ specific primers, e.g., after about 1-15 rounds of amplification using the 5′ and 3′ specific primers.

The 5′ and 3′ universal primers and the 5′ and 3′ specific primers can be used in any suitable ratio, provided that the concentration of the 5′ and 3′ universal primers equals to or is higher than the concentration of the 5′ and 3′ specific primers, respectively, when the multiplex PCRs are conducted. For example, the ratio between the 5′ and 3′ universal primers and the 5′ and 3′ specific primers can be from about 1 to about 500. The 5′ and 3′ universal primers can be used at any suitable concentration, e.g., from about 0.01 μM to about 10 μM. The 5′ and 3′ specific primers can be used at any suitable concentration, e.g., from about 0.01 μM to about 1 μM.

The universal primers and/or the specific primers can have any suitable percentage of GC content. For example, the GC content of the universal primers and/or the specific primers can be from about 30% to about 80%. Preferably, the GC content of the universal primers and/or the specific primers is from about 40% to about 60%. Normally, the difference of the GC contents of the specific sequence of the specific primers should be kept small, e.g., within about 20%.

The specific sequence of the specific primers can have any suitable Tm. For example, the Tm of the specific sequence of the specific primers can be from about 30° C. to about 80° C. and wherein the Tm is determined by the nearest neighbor method. Preferably, the Tm of the specific sequence of the specific primers is from about 40° C. to about 60° C. Normally, the difference of the Tm of specific sequence of the specific primers should be kept small, e.g., within about 20° C.

The universal primers and/or the specific primers can have any suitable length. For example, the length of the universal primers and/or the specific primers can be from about 10 nucleotides (nt) to about 40 nt. Preferably, the length of the universal primers and/or the specific primers is from about 18 nt to about 25 nt. Normally, the difference of the universal primers and/or the specific primers should be kept small, e.g., within about 10 nt.

The PCR products can be assessed by any suitable methods. For example, the PCR products can be assessed via agarose gel electrophoresis. When assessed by the agarose gel electrophoresis, the difference of the length of the PCR products is preferably more than about 30 base pairs (bp). More preferably, the difference of the length of the PCR products is from about 30 bp to about 50 bp. The PCR products can be assessed by other methods such as polyacrylamide gel electrophoresis and capillary electrophoresis.

The present methods can further comprise conducting multiplex PCR primers to amplify the target sequences using the identified optimized multiplex PCR primers. Any target sequences can be used in the optimization and/or the further amplification methods. For example, the target sequences can be of viral, bacterial, fungal, plant, animal or human origin. Preferably, the target sequences are derived from an organism that causes or is associated with a disease, e.g., a virus that causes or is associated with the severe acute respiratory syndrome (SARS-CoV).

The present methods can be used to optimize multiplex PCR primers for any suitable types of PCR. For example, the present methods can be used to optimize primers for multiplex one-step RT-PCR. In another example, the present methods can be used to optimize primers for multiplex nested PCR. In one embodiment, in the multiplex nested PCR, both the first round and second round of amplification are conventional multiplex PCR. In another embodiment, in the multiplex nested PCR, the first round of amplification is a multiplex one-step RT-PCR and the second round of amplification is a conventional multiplex PCR.

### C. COMPOSITIONS AND KITS FOR OPTIMIZING MULTIPLEX PCR PRIMERS

In another aspect, the present invention is directed to a composition for optimizing multiplex PCR primers, which composition comprises: a) a plurality of different concentrations of a plurality of 5′ and 3′ specific primers, each of said specific primers comprising a specific sequence complementary to its target sequence to be amplified and a common sequence; and b) a concentration of a 5′ and a 3′ universal primer, said 5′ universal primer being complementary to said common sequence of said 5′ specific primers and said 3′ universal primer being complementary to said common sequence of said 3′ specific primers, wherein the concentration of said 5′ and 3′ universal primer equals to or is higher than any of the concentrations of said 5′ and 3′ specific primers, respectively.

In still another aspect, the present invention is directed to a kit for optimizing multiplex PCR primers, which kit comprises: a) a plurality of 5′ and 3′ specific primers, each of said specific primers comprising a specific sequence complementary to its target sequence to be amplified and a common sequence; b) a 5′ and a 3′ universal primer, said 5′ universal primer being complementary to said common sequence of said 5′ specific primers and said 3′ universal primer being complementary to said common sequence of said 3′ specific primers; c) means for conducting a plurality of multiplex PCRs on a plurality of target sequences in the presence of said plurality of 5′ and 3′ specific primers and said 5′ and 3′ universal primers, wherein in each of said PCRS, the concentration of said 5′ and 3′ universal primers equals to or is higher than the concentration of said 5′ and 3′ specific primers, respectively, and the concentrations of said 5′ and 3′ specific primers in different PCRs are different, respectively; and d) means for assessing PCR products in said different PCRs and identifying a PCR wherein said target sequences are comparably amplified to identify optimized multiplex PCR primers for amplifying said target sequences in multiplex PCRs.

The description in the above Section B about 5′ and 3′ the universal primers, 5′ and 3′ specific primers, target sequences, etc. are equally applicable to the compositions and kits for optimizing multiplex PCR primers described in this Section C. In addition, the present methods, compositions and kits can be used to optimize multiplex PCR primers for any suitable types of PCR, e.g., standard PCR procedures, direct DNA sequencing of PCR products, ligation-mediated PCR for genomic sequencing and footprinting, molecular cloning of PCR products, enzymatic amplification of RNA by PCR (RT-PCR), cDNA amplification using one-sided (anchored) PCR and quantification of rare DNAs by PCR, etc. (See generally, Ausubel et al., (Ed.), Current Protocols in Molecular Biology, John Wiley & Sons, Inc. (2000), Chapter 15).

### D. EXAMPLES

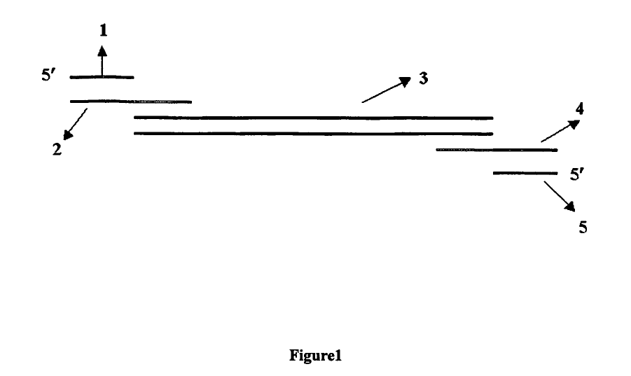

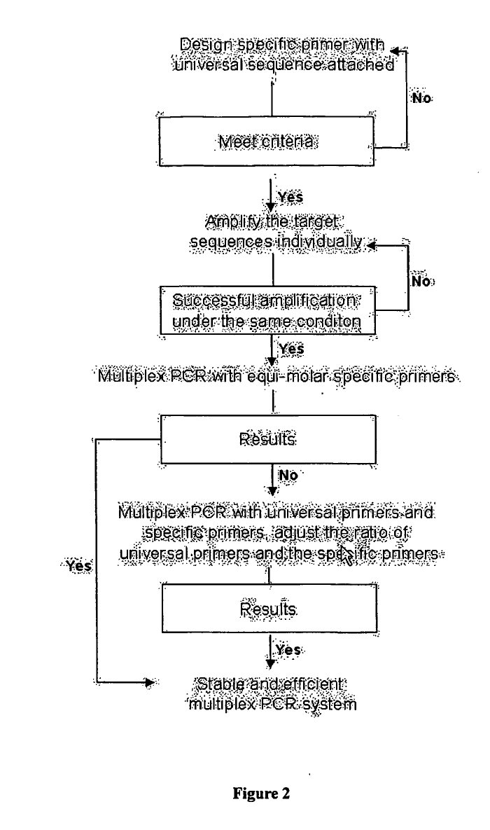

Conventional optimizing strategy for multiplex PCR program is effort and time consuming. The main problem in the optimizing multiplex PCR is the imbalance of amplification efficiency between different primers. To solve this problem, we developed an optimizing method shown in FIG. 1. A universal sequence is introduced into the 5′ end of a normal primer to form a specific primer. The universal sequence has no homology or has low homology to the target sequence and other nucleotide sequences in the target sample. Another primer is designed as a universal primer which has the same sequence as the universal sequence at the 5′ end of the specific primer. During amplification, a plurality pairs of specific primers and universal primers, and the system contains relatively high concentration of universal primers and relatively low concentration of specific primers. The optimization of multiplex PCR is achieved by adjusting the ratio of the concentration of universal primers and the concentration of specific primers. FIG. 2 shows the operation process of the invention.

### Example 1

### Optimizing HBV, HAV, HDV, and EBV Quadruple PCR

1. Reagents

Unless specifically stated, all chemical reagents in the Examples were purchased from Sigma (Woodland, Tex.). Taq DNA polymerase (MW according to DL2000) was from Tokara Co. (Dalian, PRC). dNTPs were from Shanghai BioAsia Biotechnology Co.

2. Clones of Virus Conservative Sequences

Four clones with different length (pCP10, pHAV249, pHDV142, and pEBV478) were selected from Clone library of clinical infectious pathogens from National Engineering & Research Center for Beijing Biochip Technology. The lengths of four clones are the genome length of HBV (pCP10), 249 bp (pHAV249), 142 bp (pHDV142), and 478 bp (pEBV478). These four clones can be used as template in multiplex PCR for HBV (hepatitis B virus), HAV (hepatitis A virus), HDV (hepatitis C virus) and EBV (EBV virus).

3. Primers

All primers were synthesized in Shanghai BioAsia Biotechnology Co. and purified through PAGE.

Universal primers: the upstream primer is 5′ TCA CTT GCT TCC GTT GAG G 3′ (SEQ ID NO:1), and the downstream primer is 5′ GGT TTC GGA TGT TAC AGC GT 3′ (SEQ ID NO:2). Specific primers (the underlined sequence indicates reverse self complementary and the bold are universal sequences): for hepatitis B, the upstream primer is 5° CACAGCTT TCACTTGCTTCCGTTGAGG GTT CAA GCC TCC AAG CTG TG 3′ (SEQ ID NO:3), the downstream primer is 5′ AGAACTCC GGTTTCGGATGTTACAGCGT CTG CGA GGC GAG GGA GTT CT 3′ (SEQ ID NO:4); for hepatitis A, the upstream primer is 5′ CATAGCTCACTTGCTTCCGTTGAGG TTTTGCTCCTCTTTACCATGCTATG 3′ (SEQ ID NO:5), and the downstream primer is 5′ CAAAGAGGTTTCGGATGTTACAGCGT GGAAATGTCTCAGGTACTTTCTTTG 3′ (SEQ ID NO:6); for hepatitis C, the upstream primer is 5′ACGGTCTCACTTGCTTCCGTTGAGG AACATTCCGAAGGGGACCGT 3′ (SEQ ID NO:7), and the downstream primer is 5′ CGTCCTGGTTTCGGATGTTACAGCGT GGCATCCGAAGGAGGACG 3′ (SEQ ID NO:8); and for EBV virus, the upstream primer is 5′ CATTATGTCACTTGCTTCCGTTGAGG CCCACGCGCGCATAATG 3′ (SEQ ID NO:9), and the downstream primer is 5′ CTAGGGTTTCGGATGTTACAGCGT TTCACTTCGGTCTCCCCTAG 3′ (SEQ ID NO:10).

4. Optimizing Multiplex PCR

The solution containing four viruses and four pairs of primers was first prepared and the final concentration of each primer in the solution was 50 [symbol] mol/L. The system of multiplex PCR was 25 [symbol] l and final concentration of each of the four templates (pCP10, pHAV249, pHDV142, and pEBV478) was 50 ng. The system of PCR includes 10 mmol/L of Tris-HCl (pH at 8.3 when below 24° C.), 50 mmol/L KCl, lm5 mmol/L MgCl₂, 1 unit of Taq DNA polymerase; 200 [symbol] mol/L of dNTPs; the final concentration of universal primers (upstream and downstream) at 1.0 [symbol] mol/L; nine different concentrations of specific primers (groups 1 to 9): 0.5, 0.25, 0.1, 0.05, 0.025, 0.01, 0.005, 0.0025, and 0 [symbol] mol/L. PCR reaction was performed on PTC-200 (MJ Research Inc., Miami, Fla.) and PCR cycles are predenaturing at 94° C. for 3 minute; main cycle at 94° C. for 30 sec, 55° C. for 30 sec, and 72° C. for 1 minute for 30 cycles; at 72° C. for 10 minutes; and maintaining at 4° C.

5. Electrophoresis Detection

The products were analyzed on 1.7% agarose electrophoresis with 1× TBE buffer using EmbiTec Run electrophoresis system. The electric pressure was set at 100 V and electrophoresis time as 30 minutes. Standard molecular weight sample (5 ul) DL2000 of TaKaRa (MW: 100 bp, 250 bp, 500 bp, 750 bp, 1000 bp, and 2000 bp) was loaded on the gel. PCR product (3 ul) was loaded on the gel.

6. Results

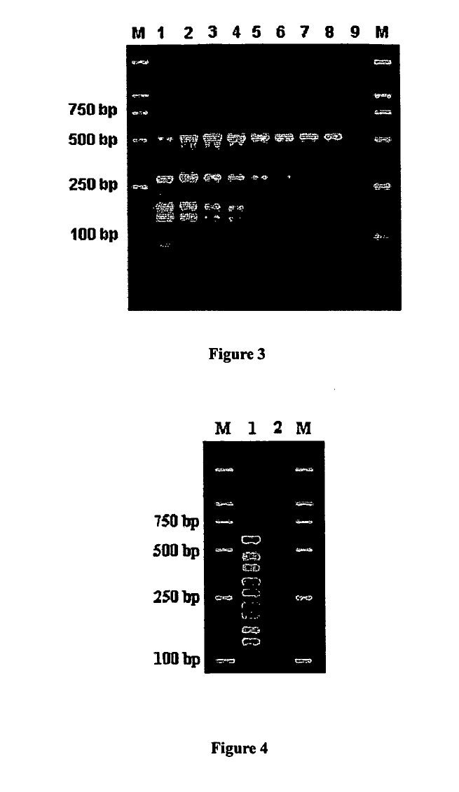

The experimental results are shown in FIG. 3. In group 2 and 4, four target fragments were amplified with relatively high efficiency and specificity, which indicated that the method of the invention significantly simplified multiplex PCR optimizing process.

### Example 2

### Optimizing Multiplex PCR of DMD Gene

1. Human DNA

All human DNA were purchased from TW-times Biotech Co.

2. Primers

All primers were synthesized in Shanghai BioAsia Biotechnology Co. and purified through PAGE.

The 5′ universal primer is 5′ TCA CTT GCT TCC GTT GAG G 3′ (SEQ ID NO:11) and the 3′ universal primer is 5′ GGT TTC GGA TGT TAC AGC GT 3′ (SEQ ID NO:12). The specific primers were designed based on the known sequences (Beggs., et al., 1990 Hum. Genet., 86, 45-48.) and are set forth in the following Table 1.

TABLE 1
Specific primers for optimizing multiplex PCR of DMD gene

| | Size | | |
|---|---|---|---|
| Exon | (bp) | No. | Sequence □5′□3′□ |
| Pm/exon | 574 | PMV_11104 | TCACTTgCTCCgTTgAggGaagatctagacagtggatacataacaaatgcatg |
| 1 | | PMV_11105 | ggTTTCggATgTTACAgCgTttctccgaaggtaattgcctcccagatctgagtcc |
| exon 3 | 449 | PMV_11106 | TCACTTgCTTCCgTTgAggtcatccatcatcttcggcagattaa |
| | | PMV_11107 | ggTTTCggATgTTACAgCgTcaggcggtagagtatgccaaatgaaaatca |
| exon 43 | 396 | PMV_11108 | TCACTTgCTTCCgTTgAgggaacatgtcaaagtcactggacttcatgg |
| | | PMV_11109 | ggTTTCggATgTTACAgCgTatatatgtgttacctacccttgtcggtcc |
| exon 50 | 310 | PMV_11110 | TCACTTgCTTCCgTTgAggcaccaaatggattaagatgttcatgaat |
| | | PMV_11111 | ggTTTCggATgTTACAgCgTtctctctcacccagtcatcacttcatag |
| exon 13 | 277 | PMV_11112 | TCACTTgCTTCCgTTgAggaataggagtacctgagatgtagcagaaat |
| | | PMV_11113 | ggTTTCggATgTTACAgCgTctgaccttaagttgttcttccaaagcag |
| exon 6 | 241 | PMV_11114 | TCACTTgCTTCCgTTgAggccacatgtaggtcaaaaatgtaatgaa |
| | | PMV_11115 | ggTTTCggATgTTACAgCgTgtctcagtaatcttcttacctatgactatgg |
| exon 47 | 220 | PMV_11116 | TCACTTgCTTCCgTTgAggcgttgttgcatttgtctgtttcagttac |
| | | PMV 11117 | ggTTTCggATgTTACAgCgTgtctaacctttatccactggagatttg |
| exon 60 | 178 | PMV_11118 | TCACTTgCTTCCgTTgAggaggagaaattgcgcctctgaaagagaacg |
| | | PMV_11119 | ggTTTCggATgTTACAgCgTctgcagaagcttccatctggtgttcagg |
| exon 52 | 152 | PMV_11120 | TCACTTgCTTCCgTTgAggaatgcaggatttggaacagaggcgtcc |
| | | PMV_11121 | ggTTTCggATgTTACAgCgTttcgatccgtaatgattgttctagcctc |

PMV_11104 to PMV_11121 correspond to SEQ ID:13 to SEQ ID:30.

3. Optimizing Multiplex PCR

One hundred (100) ng of human DNA was used as the template in a 25 μL PCR reaction system, which comprises: 10 mmol/L Tris-HCl (pH [symbol] 8.3 at 24° C.), 50 mmol/L KCl, 1.5 mmol/L MgCl₂, 1 unit Taq DNA polymerase, 200 μmol/L dNTPs, 5′ and 3′ universal primers (final concentration at 1.0 μmol/L), 9 pairs of specific primers (final concentration at 0.2 μmol/L). PCR reaction was performed on PTC-200 (MJ Research Inc., Miami, Fla.) and PCR cycles are predenaturing at 94° C. for 3 minutes; main cycle at 94° C. for 30 sec, 65° C. for 4 min, for 30 cycles; at 72° C. for 10 minutes; and maintaining at 4° C.

4. Electrophoresis Detection

The products were analyzed on 1.7% agarose electrophoresis with 1× TBE buffer using EmbiTec Run electrophoresis system. The electric pressure was set at 100 V and electrophoresis time as 30 minutes. Standard molecular weight sample (5 ul) DL2000 of TaKaRa (MW: 100 bp, 250 bp, 500 bp, 750 bp, 1000 bp, and 2000 bp) was loaded on the gel. PCR product (3 ul) was loaded on the gel.

5. Results

The experimental results are shown in FIG. 4. Amplification with relatively high efficiency and specificity were achieved with all 9 pairs of the specific primers, which indicated that the method of the invention significantly simplified multiplex PCR optimizing process.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A method for optimizing multiplex PCR primers, which method comprises:
a) providing a plurality of 5′ and 3′ specific primers, each of said specific primers comprising a specific sequence complementary to its target sequence to be amplified and a common sequence;
b) providing a 5′ and a 3′ universal primer, said 5′ universal primer being complementary to said common sequence of said 5′ specific primers and said 3′, universal primer being complementary to said common sequence of said 3′ specific primers;
c) conducting a plurality of multiplex PCRs on a plurality of target sequences in the presence of said plurality of 5′ and 3′ specific primers and said 5′ and 3′ universal primers, wherein in each of said PCRS, the concentration of said 5′ and 3′ universal primers equals to or is higher than the concentration of said 5′ and 3′ specific primers, respectively, and the concentrations of said 5′ and 3′ specific primers in different PCRs are different, respectively; and
d) assessing PCR products of said different PCRs and identifying a PCR wherein said target sequences are comparably amplified to identify optimized multiplex PCR primers for amplifying said target sequences in multiplex PCRs.

2. The method of claim 1, wherein at least one of the specific primers further comprises, at its 5′ end, a sequence complementary to a sequence at its 3′ end so that under suitable conditions, a hairpin structure is formed within the specific primer.

3. The method of claim 1, wherein the 5′ and 3′ specific primers and the 5′ and a 3′ universal primers are present in the multiplex PCRs simultaneously.

4. The method of claim 1, wherein the 5′ and 3′ universal primers are added into the multiplex PCRs after about 1-15 rounds of amplification using the 5′ and 3′ specific primers.

5. The method of claim 1, wherein the ratio between the 5′ and 3′ universal primers and the 5′ and 3′ specific primers is from about 1 to about 500.

6. The method of claim 1, wherein the concentration of the 5′ and 3′ universal primers is from about 0.01 μM to about 10 μM.

7. The method of claim 1, wherein the concentration of the 5′ and 3′ specific primers is from about 0.01 μM to about 1 μM.

8. The method of claim 1, wherein the GC content of the universal primers and/or the specific primers is from about 30% to about 80%.

9. The method of claim 8, wherein the GC content of the universal primers and/or the specific primers is from about 40% to about 60%.

10. The method of claim 1, wherein the difference of the GC contents of the specific sequence of the specific primers is within about 20%.

11. The method of claim 1, wherein the Tm of the specific sequence of the specific primers is from about 30° C. to about 80° C. and wherein the Tm is determined by the nearest neighbor method.

12. The method of claim 1, wherein the Tm of the specific sequence of the specific primers is from about 40° C. to about 60° C.

13. The method of claim 1, wherein the difference of the Tm of the specific sequence of the specific primers is within about 20° C.

14. The method of claim 1, wherein the length of the universal primers and/or the specific primers is from about 10 nucleotides (nt) to about 40 nt.

15. The method of claim 14, wherein the length of the universal primers and/or the specific primers is from about 18 nt to about 25 nt.

16. The method of claim 1, wherein the difference of the universal primers and/or the specific primers is within about 10 nt.

17. The method of claim 1, wherein the PCR products are assessed via agarose gel electrophoresis.

18. The method of claim 17, wherein the difference of the length of the PCR products is more than about 30 base pairs (bp).

19. The method of claim 18, wherein the difference of the length of the PCR products is from about 30 bp to about 50 bp.

20. The method of claim 1, wherein the PCR products are assessed via polyacrylamide gel electrophoresis and capillary electrophoresis.

21. The method of claim 1, further comprising conducting multiplex PCR primers to amplify the target sequences using the identified optimized multiplex PCR primers.

22. The method of claim 1, wherein the target sequences are of viral, bacterial, fungal, plant, animal or human origin.

23. The method of claim 1, wherein the target sequences are derived from a virus that causes or is associated with the severe acute respiratory syndrome (SARS-CoV).

24. The method of claim 1, which is used to optimize primers for multiplex one-step RT-PCR.

25. The method of claim 1, which is used to optimize primers for multiplex nested PCR.

26. The method of claim 25, wherein in the multiplex nested PCR, both the first round and second round of amplification are conventional multiplex PCR.

27. The method of claim 25, wherein in the multiplex nested PCR, the first round of amplification is a multiplex one-step RT-PCR and the second round of amplification is a conventional multiplex PCR.

28. A composition for optimizing multiplex PCR primers, which composition comprises:
a) a plurality of different concentrations of a plurality of 5′ and 3′ specific primers, each of said specific primers comprising a specific sequence complementary to its target sequence to be amplified and a common sequence; and
b) a concentration of a 5′ and a 3′ universal primer, said 5′ universal primer being complementary to said common sequence of said 5′ specific primers and said 3′ universal primer being complementary to said common sequence of said 3′ specific primers,
wherein the concentration of said 5′ and 3′ universal primer equals to or is higher than any of the concentrations of said 5′ and 3′ specific primers, respectively.

29. A kit for optimizing multiplex PCR primers, which kit comprises:
a) a plurality of 5′ and 3′ specific primers, each of said specific primers comprising a specific sequence complementary to its target sequence to be amplified and a common sequence;
b) a 5′ and a 3′ universal primer, said 5′ universal primer being complementary to said common sequence of said 5′ specific primers and said 3′ universal primer being complementary to said common sequence of said 3′ specific primers;
c) means for conducting a plurality of multiplex PCRs on a plurality of target sequences in the presence of said plurality of 5′ and 3′ specific primers and said 5′ and 3′ universal primers, wherein in each of said PCRs, the concentration of said 5′ and 3′ universal primers equals to or is higher than the concentration of said 5′ and 3′ specific primers, respectively, and the concentrations of said 5′ and 3′ specific primers in different PCRs are different, respectively; and
d) means for assessing PCR products in said different PCRs and identifying a PCR wherein said target sequences are comparably amplified to identify optimized multiplex PCR primers for amplifying said target sequences in multiplex PCRs.
